(19) United States
(12) Patent Application Publication
(10) Pub. No.: US 2026/0140079 A1
(43) Pub. Date: May 21, 2026

(54) Device for Estimating Dryness of Agricultural Products

(71) Applicant: Libin Varghese, Albertson, NY (US)

(72) Inventor: Libin Varghese, Albertson, NY (US)

(21) Appl. No.: 18/954,063

(22) Filed: Nov. 20, 2024

(51) Int. Cl. G01N 27/22; G01N 33/02

(52) U.S. Cl. CPC G01N 27/223; G01N 27/226; G01N 27/228; G01N 33/025

(57) ABSTRACT

The present invention is a device for estimating the dryness of agricultural products. Said device comprises a capacitive sensor, a microchip programmable integrated circuit, a graphic display, a dual supply system, an enclosure, a plurality of switches, and shielded cables. Said dual supply system further comprises a rechargeable battery and a solar panel. An embodiment of the present invention further comprises a mobile app for tracking recorded moisture history.

The present invention provides farmers with a reliable means to assess how dry their crops are, which is crucial for determining price, optimal storage conditions, and quality. Said device can be used alongside traditional manual drying methods, such as sun drying or smoke drying, and can also be integrated into machinery for automated use. The preferred embodiment of the present invention is a standalone, portable tool to independently measure dryness, offering flexibility and convenience for farmers and other users.

### FIELD OF THE INVENTION

The present invention relates generally to moisture content and agriculture. Particularly, the present invention is a device for estimating dryness of agricultural products comprising a capacitive sensor, a microchip programmable integrated circuit, a graphic display, a dual supply system, an enclosure, a plurality of switches, and shielded cables. Said dual supply system further comprises a rechargeable battery and a solar panel.

### BACKGROUND OF THE INVENTION

Traditionally, coconut kernels or crops are dried using methods that involve placing split coconuts in the sun for five to ten days. This drying process is labor-intensive and requires space. The dried, white flesh of a coconut is used to make coconut oil, and the price of the crop depends on its dryness. However, in many cases, farmers do not receive enough money for their crops due to dryness issues. For coconut kernels to be fully dried and ready for oil extraction, the moisture content should be approximately 5-6%, which is considered “fully dry” in agricultural terms. If the moisture percentage increases by 1%, it would result in a 1 kg loss per quintal (100 kg). Therefore, if there is a 2 kg loss per quintal, the price offered to the seller would be reduced by the estimated loss.

An objective of the present invention is to provide a device that can accurately estimate and measure the dryness of agricultural products. Farmers can use the present invention to accurately estimate the dryness of their agricultural crops. By taking a small sample of the crop from a large batch laid out for manual drying and put into the present invention, the dryness value obtained from the present invention will reflect the dryness of the entire crop laid out for drying. By ensuring the crops are properly dried, said farmers can secure a fair price for their harvest, maximizing their earnings and reducing the risk of financial loss due to moisture content discrepancies. The present invention can be calibrated based on the measured crop, increasing both accuracy and precision of recorded data.

Another objective of the present invention is to provide a device that is compatible with various crop materials. Not limited to just coconut material, the present invention can be calibrated based on the measured crop before recording data. The capacitive sensor of the present invention can accommodate different amounts of material based on the measured crop.

Another objective of the present invention is to provide a device that is portable and can be used over long periods of time. The present invention is powered by a supply supply system combining a rechargeable battery with a solar panel. The present invention can be used both indoors and outdoors, while reducing reliance on external power sources.

### SUMMARY OF THE INVENTION

The present invention is a device for estimating the dryness of agricultural products. Said device comprises a capacitive sensor, a microchip programmable integrated circuit, a graphic display, a dual supply system, an enclosure, a plurality of switches, and shielded cables. Said dual supply system further comprises a rechargeable battery and a solar panel. An embodiment of the present invention further comprises a mobile app for tracking recorded moisture history.

The present invention provides farmers with a reliable means to assess how dry their crops are, which is crucial for determining price, optimal storage conditions, and quality. Said device can be used alongside traditional manual drying methods, such as sun drying or smoke drying, and can also be integrated into machinery for automated use. The preferred embodiment of the present invention is a standalone, portable tool to independently measure dryness, offering flexibility and convenience for farmers and other users.

### BRIEF DESCRIPTION OF THE DRAWINGS

FIG. 1 is a front top right perspective view of the present invention.

FIG. 2 is a front bottom right perspective view of the present invention.

FIG. 3 is a rear top left perspective view of the present invention.

FIG. 4 is a rear bottom left perspective view of the present invention.

FIG. 5 is a front elevational view of the present invention.

FIG. 6 is a rear elevational view of the present invention.

FIG. 7 is a right elevational view of the present invention.

FIG. 8 is a left elevational view of the present invention.

FIG. 9 is a top plan view of the present invention.

FIG. 10 is a bottom plan view of the present invention.

FIG. 11 is a block diagram depicting the present invention.

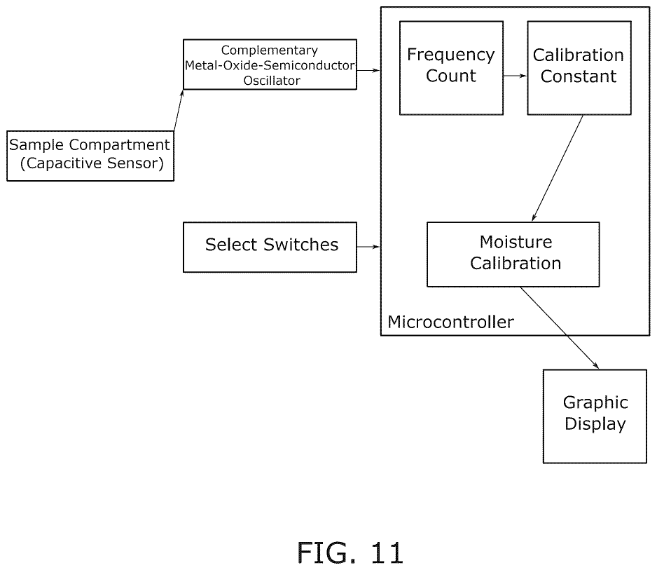

### DETAILED DESCRIPTION OF THE INVENTION

All illustrations of the drawings are for the purpose of describing selected versions of the present invention and are not intended to limit the scope of the present invention.

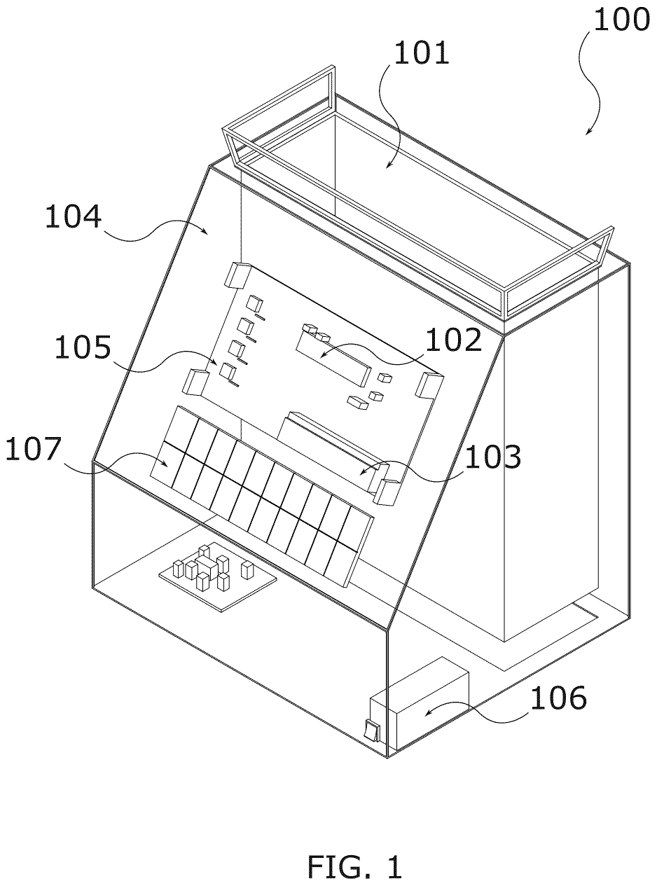

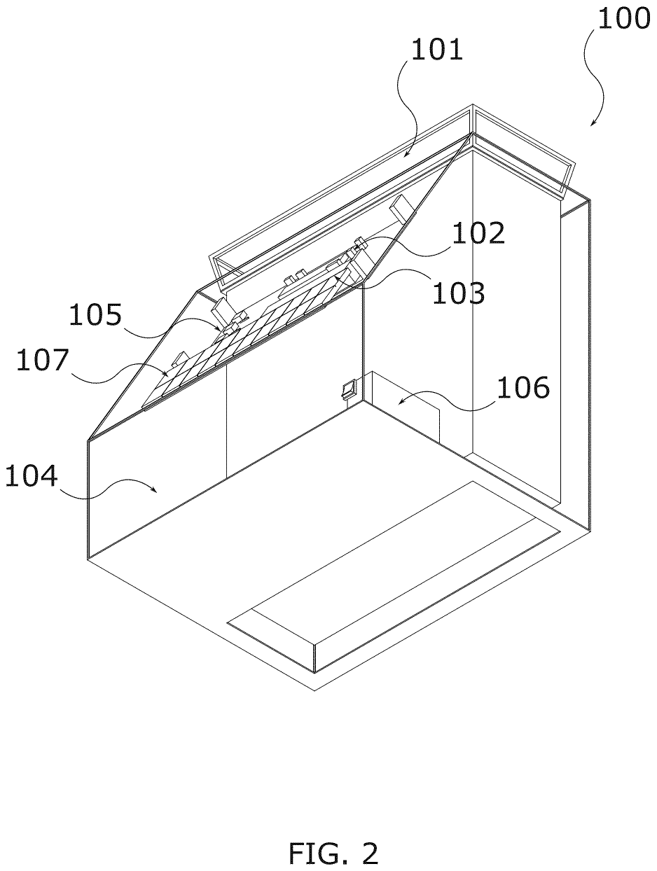

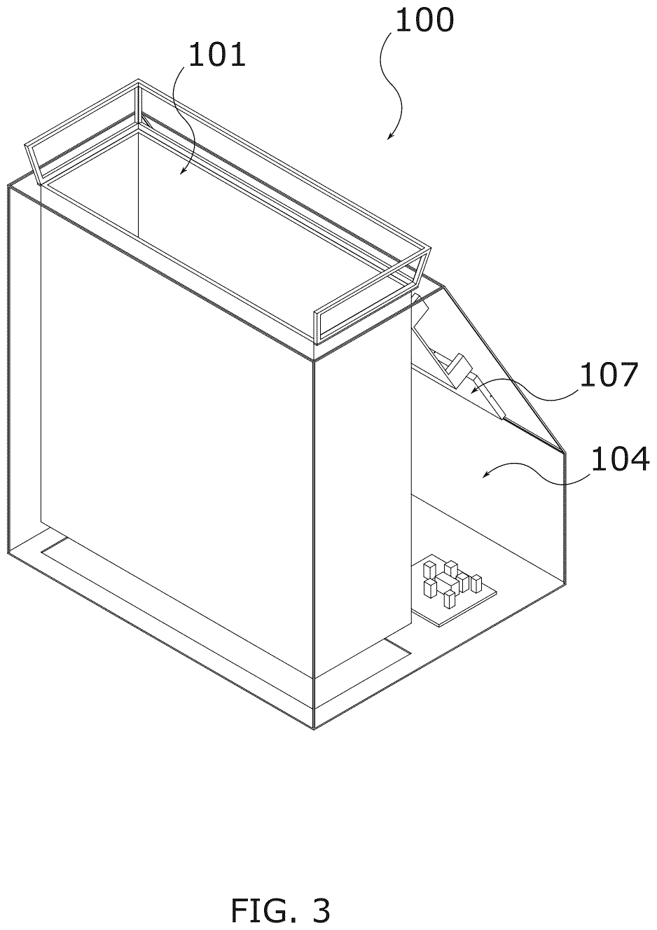

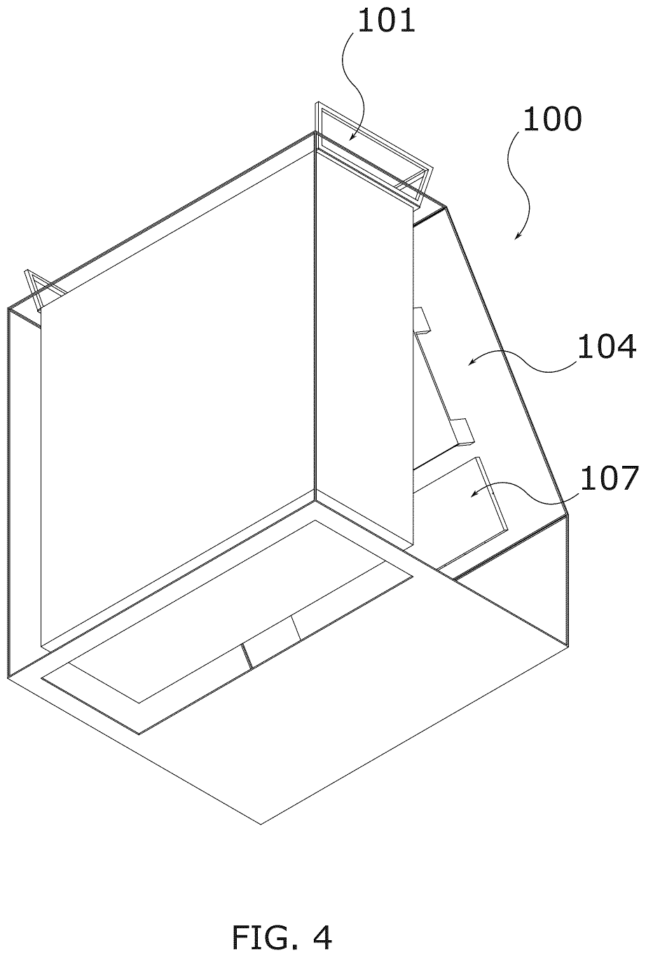

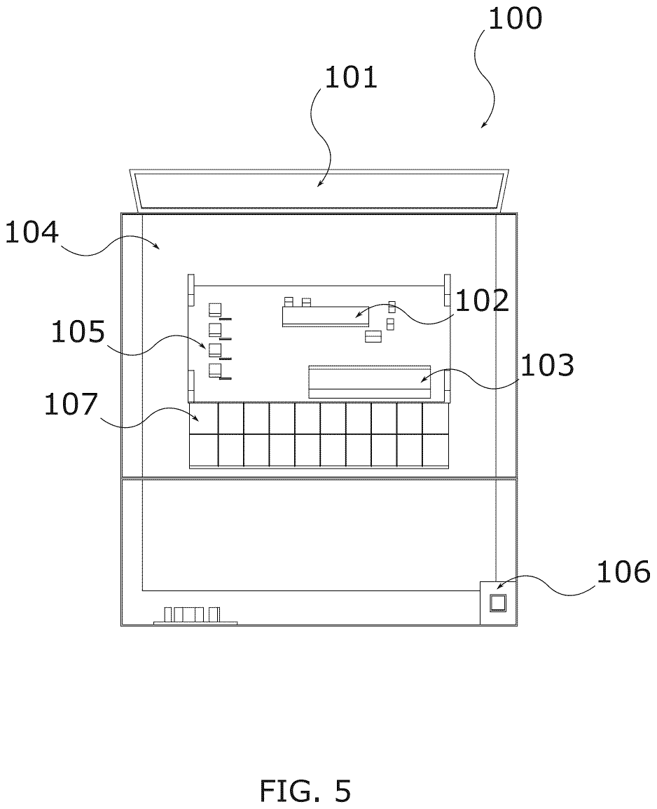

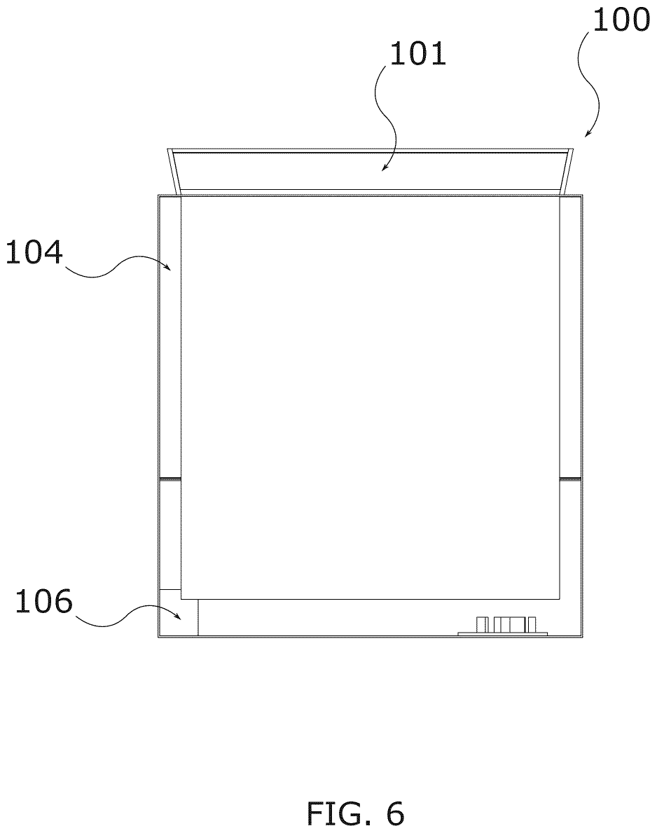

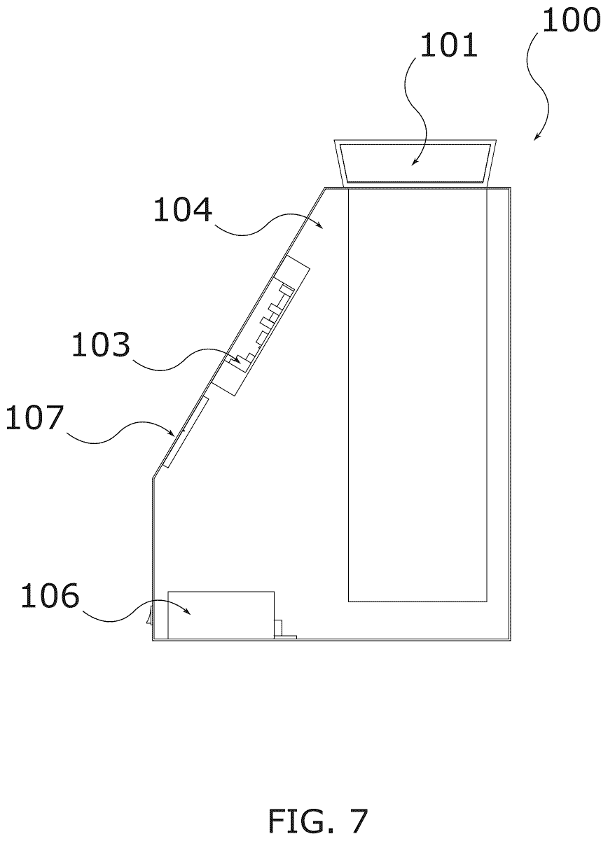

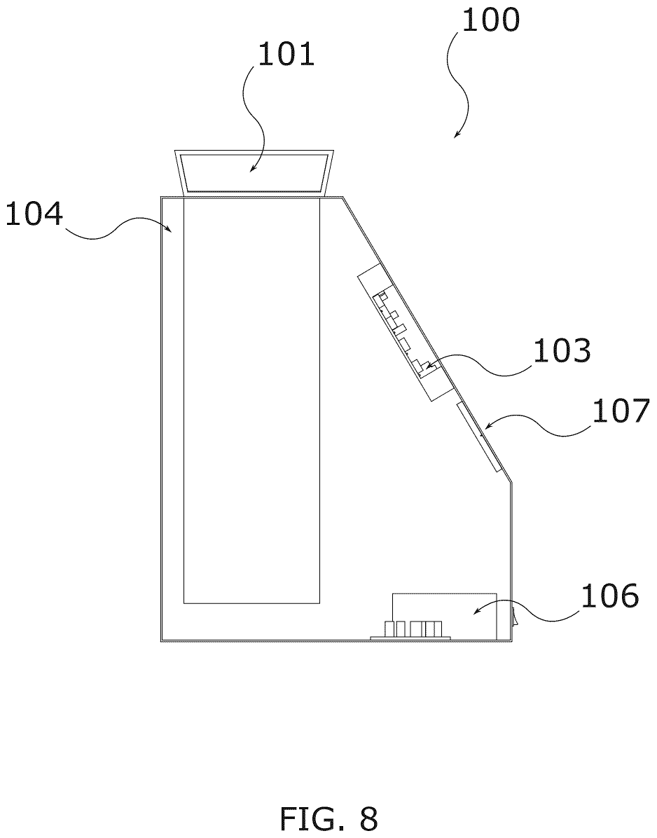

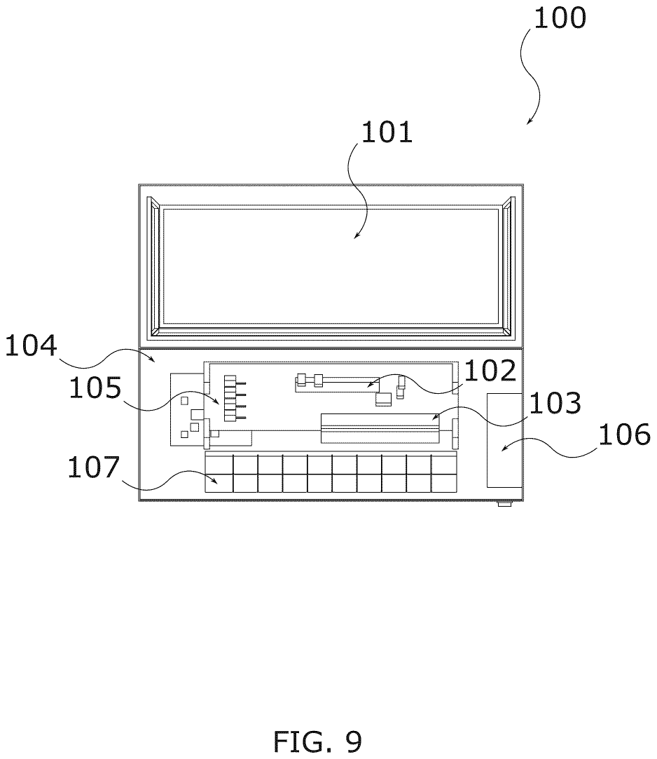

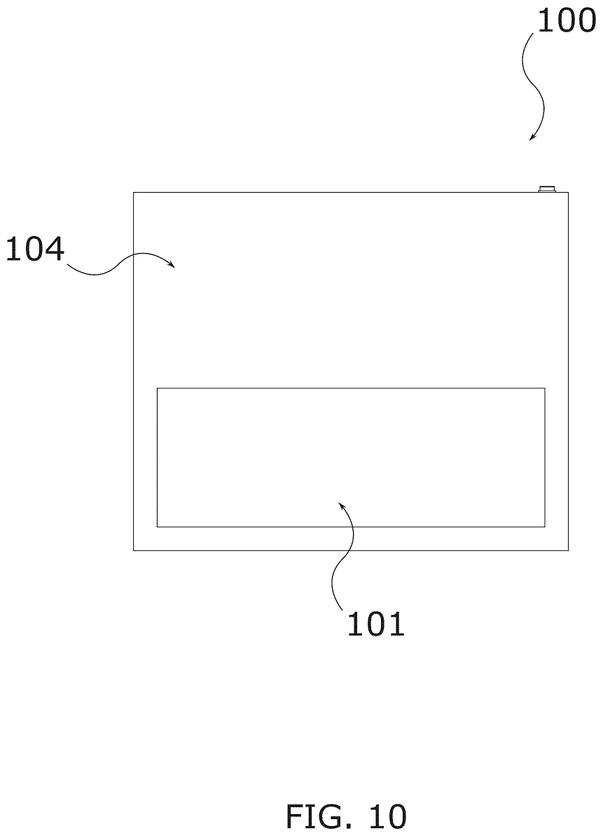

As shown in FIG. 1-10, the present invention is a device 100 for estimating the dryness of agricultural products. Said device 100 comprises a capacitive sensor 101, a microchip programmable integrated circuit 102, a graphic display 103, a dual supply system, an enclosure 104, a plurality of switches 105, and shielded cables. Said dual supply system further comprises a rechargeable battery 106 and a solar panel 107. An embodiment of the present invention further comprises a mobile app for tracking recorded moisture history. The components of the present invention work together to give a comprehensive and efficient moisture measurement system, providing reliable data and user-friendly operation for agricultural applications.

The present invention operates by using the change in capacitance as a deterministic technique to measure the dryness of crop. This measurement is successful in many crops, such as cardamom, pepper, and copra, through said device 100. Using a frequency count method, if two known moisture values are available, any unknown moisture value can be interpolated by plotting a linear graph. The known moisture values can be calibrated by determining the highest frequency value using a wet sample and the lowest frequency value using lab-measured ideal crop material. By plotting the data on a frequency-moisture graph, the frequencies for 100% moisture and 0% moisture are determined. The slope, known as the calibration constant, is derived from the linear graph.

Calibration constant (a) can be determined as follows:

$$a = ( {fc}_{2} - {fc}_{1} ) / ( {MC}_{wet} - {MC}_{dry} )$$

wherein fc₁ is the frequency (kHz) of a wet sample value, fc₂ is the frequency (kHz) of a dry sample value, MC_{wet} is the moisture content value (%) of a wet reference sample, and MC_{dry} is the moisture content value (%) of a dry reference sample.

Using the calibration constant, frequencies for 0% (f₀) and 100% (f₁₀₀) moisture content can be determined as follows:

$${f}_{100} = {fc}_{2} - ( ( 100 - {MC}_{dry} ) * a )$$

$${f}_{0} = {fc}_{2} + ( {MC}_{dry} * a )$$

Measured moisture percentage (M %) of an unknown sample can then be calculated as follows:

$$M \% = 100 / ( ( {f}_{100} - {f}_{0} ) / ( {fc}_{freq} - {f}_{0} ) )$$

wherein fc_{freq} is the measured frequency of an unknown sample.

This method of measuring frequency has proven more stable than other methods of measuring frequency in agricultural product, as it deals with high-frequency waves rather than distorted low-frequency waves. A square pulse from a complementary metal-oxide-semiconductor oscillator and a reference square pulse with a 1-second period are given to a logic gate (AND gate). The output of said AND gate, corresponding to square pulses of 1 second, is sent to a counter to calculate the number of pulses in 1 second, determining frequency.

Said capacitive sensor 101 can accommodate different sizes and amounts of material depending on the user's requirements. For example, a user can decide to measure 500 grams or 1 kilogram of material using said capacitive sensor 101. Said capacitive sensor 101 is necessary for measuring the dielectric constant of the material to determine moisture content. Said capacitive sensor 101 further comprises a sample compartment of two parallel plates with a fixed separation. The agricultural product is placed between said parallel plates. The dielectric constant of the agricultural product varies based on the moisture content of the material, affecting the overall measured capacitance. Said sample compartment is constructed from a two-sided copper-coated printed circuit board (PCB) material. The material between the thin copper layers of said sample compartment is non-conductive, allowing for the easy fabrication of structures such as electrodes. Each of said parallel plates features four horizontal electrode stripes, each spaced differently, on the inner sides. Said electrode stripes can be coated with a non-conductive protective layer, such as ceramic or plastic, enabling contactless and noninvasive measurements of abrasive or aggressive crop materials. The outer sides of said parallel plates include solid copper layers maintained at electric ground potential, serving as guard electrodes that create well-defined electric fields and reproducible measurement conditions. Said sample compartment is designed to facilitate investigations with variable lengths between said parallel plates, with the inner electrodes connected via said shielded cables.

Said microchip programmable integrated circuit 102 processes the frequency data from said capacitive sensor 101. Said microchip programmable integrated circuit 102 manages the conversion of capacitance changes into measurable frequencies and performs calculations to determine moisture content. Said microchip programmable integrated circuit 102 is designed for flexibility and programmability, allowing for updates and adjustments to the present invention's algorithms and calibration settings. Said microchip programmable integrated circuit 102 further comprises an integrated complementary metal-oxide-semiconductor oscillator. Said oscillator generates a frequency that is utilized to measure as a function of change in capacitance of the agricultural product. As the capacitance changes due to varying moisture content, said oscillator's frequency also changes. Said microchip programmable integrated circuit 102 then detects and measures this change of frequency. Said microchip programmable integrated circuit 102 analyzes the change of frequency to calculate the moisture content based on pre-established calibration constants.

To calibrate the present invention, reference samples of agricultural product, as both dry samples and wet samples, are evaluated by the user. Reference samples with known moisture contents set the upper and lower frequency limits of the agricultural product. Alternatively, in some embodiments, the present invention comprises calibration value presets. Calibration of the present invention helps to define a calibration constant that correlates frequency changes with moisture content. By comparing the calibration data with the measured frequency of an unknown sample, the device 100 calculates and displays the moisture percentage on said graphic display 103.

Said graphic display 103 is a high resolution screen that provides users with numerical values, historical data, and graphs of moisture measurements. Said graphic display 103 also provides instructions and feedback for user operations, making the system user-friendly and informative.

Said enclosure 104 protects the internal components of said device 100. In the preferred embodiment of the present invention, said enclosure 104 is made from lightweight fiber material. Said enclosure 104 is designed to be robust and durable, while the lightweight nature of said enclosure 104 ensures portability and case of handling.

Said plurality of switches 105 provide a variety of mechanisms that a user can interact with said device 100. In the preferred embodiment of the present invention, said plurality of switches 105 further comprises a push-button switch for powering said device 100 on and off. Said device 100 is programmed to enter a sleep mode to conserve energy when said device 100 is not in use. In the preferred embodiment of the present invention, said plurality of switches 105 further comprises four selection switches, identified as S1, S2, S3, and S4. Said S1 switch is used to increment and set the moisture content to the wet value. Said S2 switch decrements and sets the moisture content to the dry value. Said S3 switch allows the user to save the frequency for the corresponding moisture content of the wet sample. Said S4 switch allows the user to save the frequency for the corresponding moisture content of the dry sample. Said plurality of switches 105 are designed with user convenience and reliability in mind, making it easy to navigate through the calibration process and ensuring a seamless experience while measuring the dryness of the crop. Using said plurality of switches 105 with said graphic display 103, users can manually set reference frequences and view results of testing. This usability enables the present invention to be practical for field use.

Said shielded cables are found within said device 100. Said shielded cables are shielded to prevent noise and other electromagnetic interferences, ensuring accurate measurements and stable performance of said device 100 by minimizing signal disruption.

Said dual supply system further comprises a rechargeable battery 106 and a solar panel 107. In the preferred embodiment of the present invention, said rechargeable battery 106 is a rechargeable 9 volt battery that provides power to said device 100. Said rechargeable battery 106 is intended to ensure that said device 100 operates efficiently over extended periods of time. Said solar panel 107 is used for charging said rechargeable battery 106, further enhancing said device's 100 sustainability and reducing dependency on other external power sources.

To operate said device 100, users begin by turning on said device 100 using a switch. LCD instructions appear on said graphic display 103 to alert the user that said device 100 is working properly. Users calibrate said device 100 by using a reference sample of known moisture content, based on laboratory methods or user experience, or select the calibration value presets stored in said device 100. Once said device 100 is calibrated, users place the unknown crop sample into the sample compartment of said capacitive sensor 101. The crop sample can be preferably shredded and packed tightly to ensure accuracy. Said device 100 then measures the unknown crop sample and displays the calculated moisture content of the unknown crop sample on said graphic display 103. Users can view the results as numerical values or graphs on said graphic display 103. After measurement, users remove the sample from the sample compartment of said capacitive sensor 101.

An example use case of the present invention is described as follows. First, the user places a reference sample of highly wet kernel of coconut, shredded and tightly packed, into the sample compartment of said capacitive sensor 101. Using said S1 switch, the user can increment the value shown on said graphic display 103 to match a known moisture percentage of wet sample. The user saves the frequency value corresponding to the wet reference sample using said S3 switch. This wet sample frequency value would typically fall within a higher range of kHz. After removing the wet reference sample, the user places a reference sample of dried kernel of coconut into the sample compartment of said capacitive sensor 101. Said graphic display 103 should display a lower frequency value than the dry sample's frequency value. The user can decrement the value shown on said graphic display 103 to match a known moisture percentage of dry sample using said S2 switch. The corresponding frequency is saved using said S4 switch. With calibration complete, the user can now test any sample of coconut product.

An embodiment of the present invention further comprises a mobile app for tracking recorded moisture history. Users can transfer readings and measurements to said mobile app using GSM/Bluetooth capabilities. Said mobile app can save records of sample size, values, and timestamps. By adding and saving additional data, users can utilize said mobile app to determine average data values to increase accuracy of the present invention's usage over time.

Although the invention has been explained in relation to its preferred embodiment, it is to be understood that many other possible modifications and variations can be made without departing from the spirit and scope of the invention.

What is claimed is:

1. A device comprising:
a capacitive sensor;
a microchip programmable integrated circuit;
a graphic display;
a battery;
an enclosure;
a plurality of switches, and
cables.

2. The device of claim 1:
wherein said capacitive sensor further comprises a sample compartment;
two parallel plates; and
a plurality of electrode stripes.

3. The device of claim 1, further comprising:
a solar panel;
wherein said battery is rechargeable; and
wherein said solar panel recharges said battery.

4. The device of claim 1, further comprising:
a mobile application,
wherein said mobile application can receive and store data sent through GSM or Bluetooth communications.

5. The device of claim 1, further comprising:
calibration value presets.

6. The device of claim 1,
wherein said plurality of switches further comprises:
a switch for turning said device on or off;
a switch for incrementing a measured moisture content value;
a switch for decrementing a measured moisture content value;
a switch for saving a frequency for a corresponding moisture content value of a dry sample; and
a switch for saving a frequency for a corresponding moisture content value of a wet sample.

7. The device of claim 1,
wherein said cables are shielded cables.

8. A device comprising:
a capacitive sensor;
a sample compartment
two parallel plates;
a plurality of electrode stripes;
a microchip programmable integrated circuit;
a graphic display;
a battery;
an enclosure;
a plurality of switches, and
shielded cables.

9. The device of claim 8, further comprising:
a solar panel;
wherein said battery is rechargeable; and
wherein said solar panel recharges said battery.

10. The device of claim 8, further comprising:
a mobile application,
wherein said mobile application can receive and store data sent through GSM or Bluetooth communications.

11. The device of claim 8, further comprising:
calibration value presets.

12. The device of claim 8,
wherein said plurality of switches further comprises:
a switch for turning said device on or off;
a switch for incrementing a measured moisture content value;
a switch for decrementing a measured moisture content value;
a switch for saving a frequency for a corresponding moisture content value of a dry sample; and
a switch for saving a frequency for a corresponding moisture content value of a wet sample.

13. A device comprising:
a capacitive sensor;
a sample compartment
two parallel plates;
a plurality of electrode stripes;
a microchip programmable integrated circuit;
a graphic display;
a rechargeable battery;
a solar panel;
an enclosure;
a plurality of switches, and
shielded cables;
wherein said solar panel recharges said rechargeable battery.

14. The device of claim 8, further comprising:
a mobile application,
wherein said mobile application can receive and store data sent through GSM or Bluetooth communications.

15. The device of claim 8, further comprising:
calibration value presets.

16. The device of claim 8,
wherein said plurality of switches further comprises:
a switch for turning said device on or off;
a switch for incrementing a measured moisture content value;
a switch for decrementing a measured moisture content value;
a switch for saving a frequency for a corresponding moisture content value of a dry sample; and
a switch for saving a frequency for a corresponding moisture content value of a wet sample.
